(19) United States
(12) Patent Application Publication
(10) Pub. No.: US 2011/0160315 A1
(43) Pub. Date: Jun. 30, 2011

(54) PROCESS OF SYNTHESIS GAS CONVERSION TO LIQUID HYDROCARBON MIXTURES USING SYNTHESIS GAS CONVERSION CATALYST AND HYDROISOMERIZATION CATALYST

(75) Inventors: Charles L. Kibby, Benicia, CA (US); Robert J. Saxton, Pleasanton, CA (US); Kandaswamy Jothimurugesan, Hercules, CA (US); Tapan K. Das, Albany, CA (US)

(73) Assignee: Chevron U.S.A. Inc.

(21) Appl. No.: 12/780,672

(22) Filed: May 14, 2010

Related U.S. Application Data
(60) Provisional application No. 61/291,230, filed on Dec. 30, 2009.

(51) Int. Cl. C07C 1/04

(52) U.S. Cl. 518/713; 518/715

(57) ABSTRACT

A process is disclosed for converting synthesis gas to a liquid hydrocarbon mixture useful as distillate fuel and/or lube base oil which is substantially free of solid wax. A synthesis gas feed is contacted with a synthesis gas conversion catalyst in an upstream bed and a hydroisomerization catalyst containing a metal promoter and an acidic component in a downstream bed within a single reactor at essentially common reaction conditions. A Fischer-Tropsch wax is formed over the synthesis gas conversion catalyst and said wax is subsequently hydroisomerized over the hydroisomerization catalyst, thereby resulting in a liquid hydrocarbon mixture having a desirable product distribution.

### BACKGROUND

1. Field

The invention relates to an improved process for converting synthesis gas to liquid hydrocarbon mixtures useful as distillate fuel and/or lube base oil by contacting the gas with multiple catalysts in a stacked bed arrangement within a single reactor.

2. Description of Related Art

The majority of combustible liquid fuel used in the world today is derived from crude oil. However, there are several limitations to using crude oil as a fuel source. For example, crude oil is in limited supply.

Alternative sources for developing combustible liquid fuel are desirable. An abundant resource is natural gas. The conversion of natural gas to combustible liquid fuel typically involves a first step of converting the natural gas, which is mostly methane, to synthesis gas, or syngas, which is a mixture of carbon monoxide and hydrogen. Fischer-Tropsch synthesis is a known means for converting syngas to higher molecular weight hydrocarbon products. Fischer-Tropsch diesel has a very high cetane number and is effective in blends with conventional diesel to reduce NOₓ and particulates emitted from diesel engines, allowing them to meet stricter emission standards.

Fischer-Tropsch synthesis is often performed under conditions which produce a large quantity of C₂₁+ wax, also referred to as “Fischer-Tropsch wax,” which must be hydroprocessed to provide distillate fuels. Often, the wax is hydrocracked to reduce the chain length, and then hydrotreated to reduce oxygenates and olefins to paraffins. Hydrocracking tends to reduce the chain length of all of the hydrocarbons in the feed. When the feed includes hydrocarbons that are already in a desired range, for example, the distillate fuel range, hydrocracking of these hydrocarbons is undesirable.

Considerably different process conditions are required for hydrocracking and hydroisomerization of Fischer-Tropsch wax using relatively acidic catalysts such as SSZ-32 or ZSM-5 than for Fischer-Tropsch synthesis. For this reason commercial Fischer-Tropsch plants require separate reactors for the Fischer-Tropsch synthesis and for the subsequent hydrocracking of the product wax, and complicated and expensive separation schemes may be required to separate solid wax from lighter products.

It would be advantageous to provide a process in which both synthesis gas conversion and product hydrocracking and hydroisomerization are combined within a single reactor at a common set of conditions.

### SUMMARY

According to one embodiment, the invention relates to a process for converting synthesis gas to liquid hydrocarbons comprising contacting a feed comprising a mixture of carbon monoxide and hydrogen with a synthesis gas conversion catalyst in an upstream bed and a hydroisomerization catalyst containing a metal promoter and an acidic component in a downstream bed downstream of the upstream bed within a single reactor at an essentially common reactor temperature and an essentially common reactor pressure, such that C₂₁₊ normal paraffins are formed over the synthesis gas conversion catalyst and said C₂₁₊ normal paraffins are hydroisomerized over the hydroisomerization catalyst, thereby resulting in liquid hydrocarbons containing no greater than 5 weight % C₂₁₊ normal paraffins.

According to another embodiment, the invention relates to a process for converting synthesis gas to liquid hydrocarbons comprising contacting a feed comprising a mixture of carbon monoxide and hydrogen with a synthesis gas conversion catalyst in an upstream bed and a hydroisomerization catalyst containing a metal promoter and an acidic component in a downstream bed downstream of the upstream bed within a single reactor at an essentially common reactor temperature and an essentially common reactor pressure, such that C₂₁₊ normal paraffins are formed over the synthesis gas conversion catalyst and said C₂₁₊ normal paraffins are hydroisomerized over the hydroisomerization catalyst, thereby resulting in liquid hydrocarbons having a cloud point no greater than 15° C.

### BRIEF DESCRIPTION OF THE DRAWINGS

FIG. 1 is a schematic diagram illustrating a process for converting synthesis gas to liquid hydrocarbons according to an embodiment of the invention.

### DETAILED DESCRIPTION

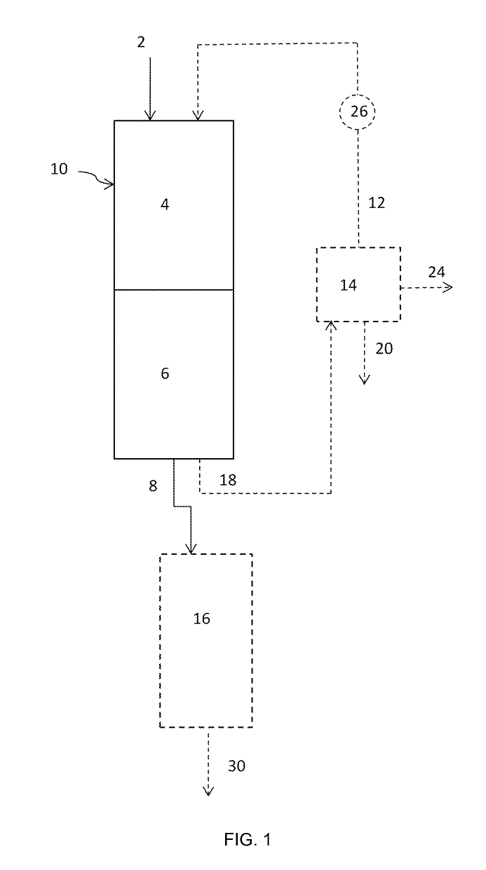

Referring to FIG. 1, a process is disclosed for the synthesis of liquid hydrocarbons in the distillate fuel and/or lube base oil range from a feed of synthesis gas 2 in a single fixed bed reactor 10. Within the fixed bed reactor, multiple, small-diameter tubes (not shown) are enclosed in a cooling medium, e.g., steam or water. Provided within the process is a method for synthesizing a mixture of olefinic and paraffinic hydrocarbons by contacting the synthesis gas with a synthesis gas conversion catalyst in a first, upstream catalyst bed 4. The hydrocarbon mixture so formed can range from methane to light wax, containing only trace amounts (<0.5 wt %) of carbon numbers above 30, and may include linear, branched and cyclic compounds. As defined herein, the terms “wax” and “solid wax” refer to C₂₁+ normal paraffins. The terms “Fischer-Tropsch wax” and “C₂₁₊ wax” are also used herein interchangeably to refer to C₂₁₊ normal paraffins. The hydrocarbon mixture is then contacted within the same reactor downstream of the first catalyst bed with a second, downstream catalyst bed 6. The downstream bed can include a hydrogenation catalyst for hydrogenating olefins and a catalyst for hydroisomerizing the straight chain hydrocarbons. The upstream bed performs synthesis gas conversion while the downstream bed performs hydroisomerization and optional hydrocracking. The synthesis gas conversion and the subsequent hydroisomerization can conveniently be carried out in a single reactor under essentially common reaction conditions without having to provide a separate reactor for hydroisomerization and optional hydrocracking. By “essentially common reaction conditions” is meant that the temperature of the cooling medium within the reactor 10 is constant from one point to another within a few degrees Celsius (e.g., 0-3° C.) and the pressure within the reactor is allowed to equilibrate between the two beds. Optionally, although not preferably, more than one cooling system may be used utilizing more than one cooling medium physically separated from each other, in which case the cooling media may be at differing temperatures. The temperatures and pressures of the upstream bed 4 and downstream bed 6 can differ somewhat, although advantageously it is not necessary to separately control the temperature and pressure of the two beds. The bed temperatures will depend on the relative exotherms of the reactions proceeding within them. Exotherms generated by synthesis gas conversion are greater than those generated by hydrocracking; therefore in the case of constant reactor tube diameter, the average upstream bed temperature will generally be higher than the average downstream bed temperature. The temperature difference between the beds will depend on various reactor design factors, including, but not limited to, the type and temperature of the cooling medium, the diameter of the tubes in the reactor, the rate of gas flow through the reactor, and so forth. For adequate thermal control, the temperatures of the two beds are preferably maintained within about 10° C. of the cooling medium temperature, and therefore the difference in temperature between the upstream and downstream beds is preferably less than about 20° C., even less than about 10° C. The pressure at the end of the upstream bed is equal to the pressure at the beginning of the downstream bed since the two beds are open to one another. Note that there will be a pressure drop from the top of the upstream bed to the bottom of the downstream bed because gas is being forced through narrow tubes within the reactor. The pressure drop across the reactor could be as high as about 50 psi (3 atm), therefore the average difference in pressure between the beds could be up to about 25 psi.

The upstream and downstream catalyst beds are arranged in series, in a stacked bed configuration. A feed of synthesis gas 2 is introduced to the reactor via an inlet (not shown). The ratio of hydrogen to carbon monoxide of the feed gas is generally high enough that productivity and carbon utilization are not negatively impacted by not adding hydrogen in addition to the hydrogen of the syngas into the reactor or producing additional hydrogen using water-gas shift. The ratio of hydrogen to carbon monoxide of the feed gas is also generally below a level at which excessive methane would be produced. Advantageously, the ratio of hydrogen to carbon monoxide is between about 1.0 and about 2.2, even between about 1.5 and about 2.2. If desired, pure synthesis gas can be employed or, alternatively, an inert diluent, such as nitrogen, CO₂, methane, steam or the like can be added. The phrase “inert diluent” indicates that the diluent is non-reactive under the reaction conditions or is a normal reaction product. It is usually advantageous to operate the syngas conversion process in a partial conversion mode, for instance 50-60 wt % based on CO, and to condense the liquid products, especially water, before either recycling the dry tail gas or sending it to an additional reactor stage. Optionally, recycle stream 18 is passed through separator 14 which utilizes a drop in temperature to condense water 20 and separate oil 24 and gas stream 12. Gas stream 12 is recycled to the reactor 10 via compressor 26. Oil 24 is optionally recycled to the reactor 10. The conversion rate drops rapidly as the partial pressures of the reactants decrease, and the water produced can damage the catalyst if its pressure gets too high. Therefore recycling the tail gas and/or staging permits operation at a low average H₂/CO ratio in the reactor, minimizing methane formation while allowing hydrogen to be used at a high ratio (e.g., at least 2.1) to form paraffinic products.

The feed gas initially contacts a synthesis gas conversion catalyst in the upstream bed 4 of the reactor.

According to one embodiment, the upstream bed contains a conventional Fischer-Tropsch synthesis gas conversion catalyst. The Fischer-Tropsch synthesis gas conversion catalyst can be any known Fischer-Tropsch synthesis catalyst. Fischer-Tropsch catalysts are typically based on group VIII metals such as, for example, iron, cobalt, nickel and ruthenium. Catalysts having low water gas shift activity and suitable for lower temperature reactions, such as cobalt, are preferred. The synthesis gas conversion catalyst can be supported on any suitable support, such as solid oxides, including but not limited to alumina, silica or titania or mixtures thereof. As nonlimiting examples, the synthesis gas conversion catalyst can be present on the support in an amount between 5% and 50% by weight in the case of cobalt, and between 0.01% and 1% by weight in the case of ruthenium.

According to another embodiment, the upstream bed contains a hybrid synthesis gas conversion catalyst. A hybrid synthesis gas conversion catalyst contains a synthesis gas conversion catalyst in combination with an olefin isomerization catalyst, for example a relatively acidic zeolite, for isomerizing double bonds in C₄⁺ olefins as they are formed. Methods for preparing a hybrid catalyst of this type are described in co-pending U.S. patent application Ser. No. 12/343,534, incorporated herein in its entirety by reference. Such a method comprises impregnating a zeolite extrudate using a solution comprising a cobalt salt to provide an impregnated zeolite extrudate and activating the impregnated zeolite extrudate by a reduction-oxidation-reduction cycle. Impregnation of a zeolite using a substantially non-aqueous cobalt solution followed by activation by a reduction-oxidation-reduction cycle reduces cobalt ion-exchange with zeolite acid sites, thereby increasing the overall activity of the zeolite component. The resulting zeolite supported cobalt catalyst comprises cobalt metal distributed as small crystallites upon the zeolite support. It should be understood that as the synthesis gas conversion catalyst activity increases, an increased amount of zeolite may be needed to produce liquid hydrocarbons having the desired product distribution. The impregnation method and the reduction-oxidation-reduction cycle used to activate the catalyst are described in detail below.

Impregnation methods followed by reduction-oxidation-reduction activation are employed for making the hybrid synthesis gas conversion catalyst (also referred to as the hybrid Fischer-Tropsch catalyst). Cobalt-ruthenium/zeolite catalysts with high activities for synthesis gas conversion to hydrocarbon liquids have been prepared using commercially available, alumina bound zeolite extrudates. With cobalt nitrate, metal loading in a single step impregnation is limited to about 6-7 weight % cobalt for these alumina bound zeolites. Thus, multiple impregnations are often needed, with intervening drying and calcination treatments to disperse and decompose the metal salts. The cobalt content was varied from 5 weight % to 15 weight %. Usually, calcination in air produced materials with lower activities than those that were formed by direct reduction of cobalt nitrate. However, direct reduction on a large scale is considered to be undesirable since it is very exothermic and it produces a pyrophoric catalyst that must then be passivated before it can be handled in air. A low temperature reduction-oxidation-reduction cycle has been found superior to a single reduction step for the activation of cobalt-ruthenium/zeolite catalysts for use as the hybrid synthesis gas conversion catalyst.

Use of zeolite extrudates has been found to be beneficial, for the relatively larger zeolite extrudate particles will cause less pressure drop within a reactor and be subject to less attrition than zeolite powder or even granular zeolite (e.g., having a particle size of about 300-1000 micrometers). Formation of particles from zeolite powder or granular zeolite plus Co/alumina and a binder, to be sized equivalent to zeolite extrudate (i.e., to avoid pressure drop and attrition) would result in blinding of cobalt sites and would probably still result in some ion exchange during the required drying and calcination steps, thus lowering the activity and selectivity of the resultant catalyst.

Methods of formation of zeolite extrudates are readily known to those of ordinary skill in the art. Wide variations in macroporosity are possible with such extrudates. For the hybrid synthesis gas conversion catalyst, without wishing to be bound by any theories, it is believed that as high a macroporosity as possible, consistent with high enough crush strength to enable operation in long reactor tubes, will be advantageous in minimizing diffusion constraints on activity and selectivity. The zeolite-mediated Fischer-Tropsch synthesis is not as diffusion-limited as that of normal Fischer-Tropsch synthesis, since the pores of the presently disclosed zeolite supported Fischer-Tropsch catalyst stay open during operation, whereas the pores of a normal Fischer-Tropsch catalyst fill with oil (melted wax).

In extrudate formation, strength is produced in a calcination step at high temperature. The temperature is high enough to cause solid state reactions between cobalt oxides and alumina or aluminosilicate portions of the material, to form very stable, essentially non-reducible phases such as spinels. Consequently, it is vital that the metal be added after the extrudate has been formed and has already undergone calcination.

As used herein, the phrases “hybrid Fischer-Tropsch catalyst” and “hybrid synthesis gas conversion catalyst” are used interchangeably to refer to a Fischer-Tropsch catalyst comprising a Fischer-Tropsch base component as well as a component containing the appropriate functionality to convert in a single-stage the primary Fischer-Tropsch products into desired products (i.e., minimize the amount of heavier, undesirable products).

As used herein, the phrase “zeolite supported cobalt catalyst” refers to catalyst wherein the cobalt metal is distributed as small crystallites upon the zeolite support. The cobalt content of the zeolite supported cobalt catalyst can depend on the alumina content of the zeolite. For example, for an alumina content of about 20 weight % to about 99 weight % based upon support weight, the hybrid synthesis gas conversion catalyst can contain, for example, from about 1 to about 20 weight % cobalt, preferably 5 to about 15 weight % cobalt, based on total catalyst weight, at the lowest alumina content. At the highest alumina content the catalyst can contain, for example, from about 5 to about 30 weight % cobalt, preferably from about 10 to about 25 weight % cobalt, based on total catalyst weight.

The hybrid synthesis gas conversion catalyst is subjected to an activation procedure comprising the steps, in sequence, of (A) reduction in hydrogen, (B) oxidation in an oxygen-containing gas, and (C) reduction in hydrogen, the activation procedure being conducted at a temperature below 500° C. It has been found that the activation procedure provides zeolite supported cobalt catalyst with improved reaction rates when the catalyst is prepared by impregnation of a zeolite support with cobalt. Moreover, the activation procedure can significantly improve activity of promoted, zeolite supported cobalt catalyst, wherein a promoter such as, for example, Ru, Rh, Pd, Cu, Ag, Au, Zn, Cd, Hg, and/or Re has been previously added to improve activity. The hybrid synthesis gas conversion catalyst is produced by subjecting a zeolite supported cobalt catalyst to an activation procedure including the steps of (i) reduction, (ii) oxidation, and (iii) reduction, herein termed “ROR activation” while under a temperature below 500° C., for example, below 450° C. By subjecting the zeolite supported cobalt catalyst to ROR activation, the activity of the resultant catalyst can be increased by as much as about 100%.

A zeolite support is a molecular sieve that contains silica in the tetrahedral framework positions. Examples include, but are not limited to, silica-only (silicates), silica-alumina (aluminosilicates), silica-boron (borosilicates), silica-germanium (germanosilicates), alumina-germanium, silica-gallium (gallosilicates) and silica-titania (titanosilicates), and mixtures thereof.

Molecular sieves, in turn, are crystalline materials that have regular passages (pores). If examined over several unit cells of the structure, the pores will form an axis based on the same units in the repeating crystalline structure. While the overall path of the pore will be aligned with the pore axis, within a unit cell, the pore may diverge from the axis, and it may expand in size (to form cages) or narrow. The axis of the pore is frequently parallel with one of the axes of the crystal. The narrowest position along a pore is the pore mouth. The pore size refers to the size of the pore mouth. The pore size is calculated by counting the number of tetrahedral positions that form the perimeter of the pore mouth. A pore that has 10 tetrahedral positions in its pore mouth is commonly called a 10-ring pore. Pores of relevance to catalysis in this application have pore sizes of 8 rings or greater. If a molecular sieve has only one type of relevant pore with an axis in the same orientation to the crystal structure, it is called 1-dimensional. Molecular sieves may have pores of different structures or may have pores with the same structure but oriented in more than one axis related to the crystal. In these cases, the dimensionality of the molecular sieve is determined by summing the number of relevant pores with the same structure but different axes with the number of relevant pores of different shape.

Exemplary zeolite supports of the hybrid synthesis gas conversion catalyst include, but are not limited to, those designated SSZ-13, SSZ-33, SSZ-46, SSZ-53, SSZ-55, SSZ-57, SSZ-58, SSZ-59, SSZ-64, ZSM-5, ZSM-11, ZSM-12, TS-1, MTT (e.g., SSZ-32, ZSM-23 and the like), H—Y, BEA (zeolite Beta), SSZ-60 and SSZ-70. These molecular sieves each contain silicon as the major tetrahedral element, have 8 to 12 ring pores, and are microporous molecular sieves, meaning having pore mouths of 20 rings or less.

The zeolite supports can have an External Surface Area of between about 100 m²/g and about 300 m²/g, for example, about 180 m²/g. Micropore values for 80% ZSM-5 should be between about 90 and 112 μL/g, with lower values implying some occlusion or loss of micropore structure. BET Surface Area is a sum of external area and micropore area (more properly calculated as a volume). The zeolite supports can further have Porosity of between about 30 and 80%, Total Intrusion Volume of between about 0.25 and 0.60 cc/g, and Crush Strength of between about 1.25 and 5 lb/mm. Si/Al ratio (for zeolite component only) can be between about 10 and 100.

A promoter, such as ruthenium or the like may be included in the hybrid synthesis gas conversion catalyst if desired. For a catalyst containing about 10 weight % cobalt, the amount of ruthenium can be from about 0.01 to about 0.50 weight %, for example, from about 0.05 to about 0.25 weight % based upon total catalyst weight. The amount of ruthenium would accordingly be proportionately higher or lower for higher or lower cobalt levels, respectively. A catalyst level of about 10 weight % has been found to best for 80 weight % ZSM-5 and 20 weight % alumina. The amount of cobalt can be increased as amount of alumina increases, up to about 20 weight % Co.

The ROR activation procedure of the present disclosure may be used to improve activity of the hybrid synthesis gas conversion catalyst of the present disclosure. Thus, any technique well known to those having ordinary skill in the art to distend the catalytic metals in a uniform manner on the catalyst zeolite support is suitable, assuming they do not promote ion exchange with zeolite acid sites.

The method employed to deposit catalytic metals onto the zeolite support can involve an impregnation technique using a substantially non-aqueous solution containing soluble cobalt salt and, if desired, a soluble promoter metal salt, e.g., ruthenium salt, in order to achieve the necessary metal loading and distribution required to provide a highly selective and active hybrid synthesis gas conversion catalyst.

Initially, the zeolite support can be treated by oxidative calcination at a temperature in the range of from about 450° to about 900° C., for example, from about 600° to about 750° C. to remove water and any organics from the zeolite support.

Meanwhile, non-aqueous organic solvent solution of a cobalt salt, and, if desired, aqueous or non-aqueous organic solvent solutions of ruthenium salts, for example, are prepared. Any suitable ruthenium salt, such as ruthenium nitrate, chloride, acetate or the like can be used. Aqueous solutions for the promoters can be used in very small amounts. As used herein, the phrase “substantially non-aqueous” refers to a solution that includes at least 95 volume % non-aqueous solution. In general, any metal salt which is soluble in the organic solvent and will not have a poisonous effect on the catalyst can be utilized.

The non-aqueous organic solvent is a non-acidic liquid which is formed from moieties selected from the group consisting of carbon, oxygen, hydrogen and nitrogen, and possesses a relative volatility of at least 0.1. The phrase “relative volatility” refers to the ratio of the vapor pressure of the solvent to the vapor pressure of acetone, as reference, when measured at 25° C.

It has been found that an aqueous solution containing a cobalt salt migrates into the microporous hydrophilic zeolite, and the cobalt replaces zeolite acid sites. In contrast, use of a non-aqueous solution containing a cobalt salt minimizes replacement of zeolite acid sites with metal. In particular, when cobalt ions in solution exchange with acidic protons in the zeolite, the cobalt ions essentially titrate the acidic sites, since the ability of the cobalt ion to promote acid-catalyzed reactions is much less than that of the protons they displace. This would not matter if the cobalt in those positions were easy to reduce during catalyst activation, because reduction by hydrogen during that process would regenerate the proton acidity according to the equation: Co⁺²+H₂=Co⁰+2H⁺. Unfortunately, the ion exchange sites are quite stable positions for cobalt and cobalt ions there are not readily reduced during normal activation procedures. As the reduction in the amount of reducible Co also decreases the activity of the Fischer-Tropsch component in the catalyst, it is bad for both functions.

Suitable solvents include, for example, ketones, such as acetone, butanone(methyl ethyl ketone); the lower alcohols, e.g., methanol, ethanol, propanol and the like; amides, such as dimethyl formamide; amines, such as butylamine; ethers, such as diethylether and tetrahydrofuran; hydrocarbons, such as pentane and hexane; and mixtures of the foregoing solvents. In an embodiment, the solvents are acetone, for cobalt nitrate or tetrahydrofuran.

Suitable cobalt salts include, for example, cobalt nitrate, cobalt acetate, cobalt carbonyl, cobalt acetylacetonate, or the like. Likewise, any suitable ruthenium salt, such as ruthenium nitrate, chloride, acetate or the like can be used. In an embodiment, ruthenium acetylacetonate is used. In general, any metal salt which is soluble in the organic solvent and will not have a poisonous effect on the metal catalyst or on the acid sites of the zeolite can be utilized.

The calcined zeolite support is then impregnated in a dehydrated state with the substantially non-aqueous, organic solvent solution of the metal salts. Thus, the calcined zeolite support should not be unduly exposed to atmospheric humidity so as to become rehydrated.

Any suitable impregnation technique can be employed including techniques well known to those skilled in the art so as to distend the catalytic metals in a uniform thin layer on the catalyst zeolite support. For example, the cobalt along with the oxide promoter can be deposited on the zeolite support material by the “incipient wetness” technique. Such technique is well known and requires that the volume of substantially non-aqueous solution be predetermined so as to provide the minimum volume which will just wet the entire surface of the zeolite support, with no excess liquid. Alternatively, the excess solution technique can be utilized if desired. If the excess solution technique is utilized, then the excess solvent present, e.g., acetone, is merely removed by evaporation.

Next, the substantially non-aqueous solution and zeolite support are stirred while evaporating the solvent at a temperature of from about 25° to about 50° C. until “dryness”.

The impregnated catalyst is slowly dried at a temperature of from about 110° to about 120° C. for a period of about 1 hour so as to spread the metals over the entire zeolite support. The drying step is conducted at a very slow rate in air.

The dried catalyst may be reduced directly in hydrogen or it may be calcined first. In the case of impregnation with cobalt nitrate, direct reduction can yield a higher cobalt metal dispersion and synthesis activity, but reduction of nitrates is difficult to control and calcination before reduction is safer for large scale preparations. Also, a single calcination step to decompose nitrates is simpler if multiple impregnations are needed to provide the desired metal loading. Reduction in hydrogen requires a prior purge with inert gas, a subsequent purge with inert gas and a passivation step in addition to the reduction itself, as described later as part of the ROR activation. However, impregnation of cobalt carbonyl must be carried out in a dry, oxygen-free atmosphere and it must be decomposed directly, then passivated, if the benefits of its lower oxidation state are to be maintained.

The dried catalyst is calcined by heating slowly in flowing air, for example 10 cc/gram/minute, to a temperature in the range of from about 200° to about 350° C., for example, from about 250° to about 300° C., that is sufficient to decompose the metal salts and fix the metals. The aforesaid drying and calcination steps can be done separately or can be combined. However, calcination should be conducted by using a slow heating rate of, for example, 0.5° to about 3° C. per minute or from about 0.5° to about 1° C. per minute and the catalyst should be held at the maximum temperature for a period of about 1 to about 20 hours, for example, for about 2 hours.

The foregoing impregnation steps are repeated with additional substantially non-aqueous solutions in order to obtain the desired metal loading. Ruthenium and other promoter metal oxides are conveniently added together with cobalt, but they may be added in other impregnation steps, separately or in combination, either before, after, or between impregnations of cobalt.

After the last impregnation sequence, the loaded catalyst zeolite support is then subjected to the ROR activation treatment. The ROR activation treatment of the present disclosure must be conducted at a temperature considerably below 500° C. in order to achieve the desired increase in activity and selectivity of the cobalt-impregnated hybrid synthesis gas conversion catalyst. Temperatures of 500° C. or above reduce activity and liquid hydrocarbon selectivity of the cobalt-impregnated catalyst. Suitable ROR activation temperatures are below 500° C., preferably below 450° C. and most preferably, at or below 400° C. Thus, ranges of 100° or 150° to 450° C., for example, 250° to 400° C. are suitable for the reduction steps. The oxidation step should be limited to 200° to 300° C. These activation steps are conducted while heating at a rate of from about 0.1° to about 5° C., for example, from about 0.1° to about 2° C.

The impregnated catalyst can be slowly reduced in the presence of hydrogen. If the catalyst has been calcined after each impregnation, to decompose nitrates or other salts, then the reduction may be performed in one step, after an inert gas purge, with heating in a single temperature ramp (e.g., 1° C./min.) to the maximum temperature and held at that temperature, from about 250° or 300° to about 450° C., for example, from about 350° to about 400° C., for a hold time of 6 to about 65 hours, for example, from about 16 to about 24 hours. Pure hydrogen is preferred in the first reduction step. If nitrates are still present, the reduction is best conducted in two steps wherein the first reduction heating step is carried out at a slow heating rate of no more than about 5° C. per minute, for example, from about 0.1° to about 1° C. per minute up to a maximum hold temperature of 200° to about 300° C., for example, 200° to about 250° C., for a hold time of from about 6 to about 24 hours, for example, from about 16 to about 24 hours under ambient pressure conditions. In the second treating step of the first reduction, the catalyst can be heated at from about 0.5° to about 3° C. per minute, for example, from about 0.1° to about 1° C. per minute to a maximum hold temperature of from about 250° or 300° up to about 450° C., for example, from about 350° to about 400° C. for a hold time of 6 to about 65 hours, for example, from about 16 to about 24 hours. Although pure hydrogen is preferred for these reduction steps, a mixture of hydrogen and nitrogen can be utilized.

Thus, the reduction may involve the use of a mixture of hydrogen and nitrogen at 100° C. for about one hour; increasing the temperature 0.5° C. per minute until a temperature of 200° C.; holding that temperature for approximately 30 minutes; and then increasing the temperature 1° C. per minute until a temperature of 350° C. is reached and then continuing the reduction for approximately 16 hours. Reduction should be conducted slowly enough and the flow of the reducing gas maintained high enough to maintain the partial pressure of water in the off-gas below 1%, so as to avoid excessive steaming of the exit end of the catalyst bed. Before and after all reductions, the catalyst must be purged in an inert gas such as nitrogen, argon or helium.

The reduced catalyst is passivated at ambient temperature (25°-35° C.) by flowing diluted air over the catalyst slowly enough so that a controlled exotherm of no larger than +50° C. passes through the catalyst bed. After passivation, the catalyst is heated slowly in diluted air to a temperature of from about 300° to about 350° C. (preferably 300° C.) in the same manner as previously described in connection with calcination of the catalyst.

The temperature of the exotherm during the oxidation step should be less than 100° C., and will be 50-60° C. if the flow rate and/or the oxygen concentration are dilute enough. If it is even less, the oxygen is so dilute that an excessively long time will be needed to accomplish the oxidation. There is a danger in exceeding 300° C. locally, since cobalt oxides interact with alumina and silica at temperatures above 400° C. to make irreducible spinels, and above 500° C., Ru makes volatile, highly toxic oxides.

Next, the reoxidized catalyst is then slowly reduced again in the presence of hydrogen, in the same manner as previously described in connection with the initial reduction of the impregnated catalyst. This second reduction is much easier than the first. Since nitrates are no longer present, this reduction may be accomplished in a single temperature ramp and held, as described above for reduction of calcined catalysts.

The hybrid synthesis gas conversion catalyst has an average particle diameter, which depends upon the type of reactor to be utilized, of from about 0.01 to about 6 millimeters; for example, from about 1 to about 6 millimeters for a fixed bed; and for example, from about 0.01 to about 0.11 millimeters for a reactor with the catalyst suspended by gas, liquid, or gas-liquid media (e.g., fluidized beds, slurries, or ebullating beds).

According to yet another embodiment, the upstream bed 4 contains a mixture of conventional Fischer-Tropsch catalyst and a hybrid synthesis gas conversion catalyst, wherein the bed contains between about 1 and about 99 weight % conventional Fischer-Tropsch catalyst and about 1 and about 99 weight % hybrid synthesis gas conversion catalyst, based on total catalyst weight.

The downstream catalyst bed 6 contains a hydroisomerization catalyst for hydroisomerizing straight chain hydrocarbons. The hydroisomerization catalyst is a bifunctional catalyst containing a hydrogenation component comprising a metal promoter and an acidic component. The hydroisomerization catalyst can be selected from 10-ring and larger zeolites. Suitable materials for use as the hydroisomerization catalyst include, as not limiting examples, SSZ-32, ZSM-57, ZSM-48, ZSM-22, ZSM-23, SAPO-11 and Theta-1. The hydroisomerization catalysts can also be non-zeolitic materials.

According to one embodiment, the downstream catalyst bed 6 also contains a hydrocracking catalyst for cracking straight chain hydrocarbons. The hydrocracking catalyst is an acid catalyst material and can be a material such as amorphous silica-alumina or tungstated zirconia or a zeolitic or non-zeolitic crystalline medium pore molecular sieve. The phrase “medium pore” as used herein means having a crystallographic free diameter in the range of from about 3.9 to about 7.1 Angstrom when the molecular sieve is in the calcined form. The crystallographic free diameters of the channels of molecular sieves are published in the “Atlas of Zeolite Framework Types”, Fifth Revised Edition, 2001, by C H. Baerlocher, W. M. Meier, and D. H. Olson. Elsevier, pp 10-15, which is incorporated herein by reference. Examples of suitable hydrocracking medium pore molecular sieves include zeolite Y, zeolite X and the so called ultra stable zeolite Y and high structural silica:alumina ratio zeolite Y such as for example described in U.S. Pat. Nos. 4,401,556, 4,820,402 and 5,059,567, herein incorporated by reference. Small crystal size zeolite Y, such as described in U.S. Pat. No. 5,073,530, herein incorporated by reference, can also be used. Other zeolites which show utility as cracking catalysts include those designated as SSZ-13, SSZ-33, SSZ-46, SSZ-53, SSZ-55, SSZ-57, SSZ-58, SSZ-59, SSZ-64, ZSM-5, ZSM-11, ZSM-12, ZSM-23, H-Y, beta, mordenite, SSZ-74, ZSM-48, TON type zeolites, ferrierite, SSZ-60 and SSZ-70. Non-zeolitic medium pore molecular sieves which can be used include, for example, silicoaluminophosphates (SAPO) such as SAPO-11, SAPO-31 and SAPO-41, ferroaluminophosphate, titanium aluminophosphate and the various ELAPO molecular sieves described in U.S. Pat. No. 4,913,799 and the references cited therein. Details regarding the preparation of various non-zeolite molecular sieves can be found in U.S. Pat. No. 5,114,563 (SAPO); U.S. Pat. No. 4,913,799 and the various references cited in U.S. Pat. No. 4,913,799, hereby incorporated by reference in their entirety. Mesoporous molecular sieves can also be included, for example the M41S family of materials (J. Am. Chem. Soc. 1992, 114, 10834-10843), MCM-41 (U.S. Pat. Nos. 5,246,689, 5,198,203, 5,334,368), and MCM48 (Kresge et al., Nature 359 (1992) 710).

As is well known, hydrocracking and hydroisomerization catalysts can optionally contain a metal promoter and a cracking component. The metal promoter is typically a metal or combination of metals selected from Group VIII noble and non-noble metals, Group 1B coinage metals, and Group VIB metals. Noble and coinage metals which can be used include platinum, palladium, rhodium, ruthenium, osmium, silver, gold and iridium, or any combination thereof. Non-noble metals which might be used include molybdenum, tungsten, nickel, cobalt, copper, rhenium, or any combination thereof.

The metal promoter can be incorporated into the catalyst mixture by any one of numerous procedures. It can be added either to the cracking component, to the support or a combination of both. In the alternative, the Group VIII components can be added to the cracking component or matrix component by co-mulling, impregnation, or ion exchange and the Group VI components, i.e., molybdenum and tungsten can be combined with the refractory oxide by impregnation, co-mulling or co-precipitation. These components are usually added as a metal salt which can be thermally converted to the corresponding oxide in an oxidizing atmosphere or reduced to the metal with hydrogen or other reducing agent.

According to one embodiment, the downstream catalyst bed 6 contains a combination of a hydroisomerization component, e.g. a noble metal-promoted zeolite of the SSZ-32 family and a solid acid hydrocracking component, e.g. Pd/ZSM-5. The proportion of cracking and hydroisomerization catalysts in the downstream bed is advantageously optimized to balance the isomerization activity with the cracking activity. If there is excessive cracking catalyst the resulting product may be lighter than desired. The cracking catalyst converts the n-paraffin wax product to a suitable chain length while the hydroisomerization component isomerizes the n-paraffin product, resulting in an entirely liquid isomerized product. If the desire is to produce a heavier, diesel range product, then the catalyst combination should exhibit less cracking and more isomerization. By including Pd/SSZ-32, for example, it has been found that more isomerization can be achieved. If there is insufficient cracking catalyst the hydroisomerization catalyst may be unable to convert the wax to liquid products under the mild process conditions of the present process. Accordingly, it may be advantageous to include in the downstream bed a combination of both a cracking catalyst component and a hydroisomerization catalyst in the correct proportions so as to obtain a desired product, e.g. having an average molecular weight in the diesel range, i.e. C₁₁ to C₂₀, and containing no solid wax phase at ambient conditions.

The amounts of hydrocracking and hydroisomerization catalysts in the downstream bed can be suitably varied to obtain the desired product. If the catalyst mixture amount is too low, there will be insufficient cracking and/or isomerization to convert all of the wax; whereas if there is too much catalyst mixture in the downstream bed, the resulting product may be too light. The amount of catalyst mixture needed in the downstream bed will in part depend on the tendency of the synthesis gas conversion catalyst in the upstream bed to produce wax and will in part depend on process conditions. In general, the weight of the catalyst mixture in the downstream bed is between about 0.5 and about 2.5 times the weight of the catalyst in the upstream bed.

The reaction temperature is suitably from about 160° C. to about 260° C., for example, from about 175° C. to about 250° C. or from about 185° C. to about 235° C. Higher reaction temperatures favor lighter products. The total pressure is, for example, from about 1 to about 100 atmospheres, for example, from about 3 to about 35 atmospheres or from about 5 to about 20 atmospheres. Higher reaction pressures favor heavier products. The gaseous hourly space velocity based upon the total amount of feed is less than 20,000 volumes of gas per volume of catalyst per hour, for example, from about 100 to about 5000 v/v/hour or from about 1000 to about 2500 v/v/hour.

Fixed bed reactor systems have been developed for carrying out the Fischer-Tropsch reaction. Such reactors are suitable for use in the present process. For example, suitable Fischer-Tropsch reactor systems include multi-tubular fixed bed reactors the tubes of which are loaded with the upstream and downstream catalyst beds.

The present process provides for a high yield of paraffinic hydrocarbons in the middle distillate and/or light base-oil range under essentially the same reaction conditions as the synthesis gas conversion. The hydrocarbon mixture produced 8 is liquid at about 0° C. The hydrocarbon mixture produced is substantially free of solid wax by which is meant that the product is a single liquid phase at ambient conditions without the visibly cloudy presence of an insoluble solid wax phase. By “ambient conditions” is meant a temperature of 15° C. and a pressure of 1 atmosphere. The process results in the following composition:
0-20, for example, 5-15 or 8-12, weight % CH₄;
0-20, for example, 5-15 or 8-12, weight % C₂-C₄;
60-95, for example, 70-90 or 76-84, weight % C₅₊; and
0-5 weight % C₂₁₊ normal paraffins.

In a typical Fischer-Tropsch process, the product obtained is a predominantly a normal or linear paraffin product, meaning free of branching. If the C₂₁₊ fraction present within a predominantly linear product is greater than 5 weight %, the product has been found to contain a separate, visible solid wax phase. Products of the present process may actually contain C₂₁₊ at greater than 5 weight % without a visible solid wax phase. This is believed to be because of the hydroisomerization capability of the hydroisomerization catalyst. Branched paraffins have lower melting points compared with normal or linear paraffins such that products of the present process can contain a greater percentage of C₂₁₊ fraction and still remain a liquid which is free of a separate, visible solid wax phase at ambient conditions. The present process provides a product having a concentration of isomerized (i.e., containing at least single branches) C₂₁₊ paraffin of at least 30 weight % based on the weight of the C₂₁₊ fraction (as determined by gas chromatography). The result is a product which is liquid and pourable at ambient conditions. Liquid hydrocarbons produced by the present process advantageously have a cloud point as determined by ASTM D 2500-09 of 15° C. or less, even 10° C. or less, even 5° C. or less, and even as low as 2° C.

In addition, the present process provides for a high yield of paraffinic hydrocarbons in the middle distillate and/or light base-oil range without the need for separation of products arising from the first catalyst bed and without the need for a second reactor containing catalyst for hydrocracking and/or hydroisomerization. Process water arising from the first catalyst bed is not required to be separated from the reactor during the hydroisomerization of said C₂₁₊ normal paraffins. It has been found that with a proper combination of catalyst composition, catalyst bed placement and reaction conditions, both the synthesis gas conversion reaction and the subsequent hydrocracking and/or hydroisomerization reactions can be conducted within a single reactor under essentially common process conditions.

While it is not required, under certain circumstances it may be desirable to run the present process using an optional second reactor 16 for further hydrocracking and/or hydroisomerization or to provide backup hydrocracking and/or hydroisomerization capacity, resulting in product stream 30. Also optionally, the present process can be run with the addition of makeup hydrogen (not shown).

An additional advantage to the present process is that undesired methane selectivity is kept low as a result of maintaining the process temperature in the lower end of the optimum range for Fischer-Tropsch synthesis and considerably lower than what is generally believed required for adequate hydrocracking and hydroisomerization activity.

### EXAMPLES

### Preparation of Conventional Fischer-Tropsch Synthesis Gas Conversion Catalyst Comprising 20 wt % Cobalt-0.5 wt % Ruthenium-1.0 wt % Lanthanum Oxide Supported on Alumina (“Catalyst A”)

A three-step incipient wetness impregnation method was used to prepare the Fischer-Tropsch catalyst. A solution was prepared by dissolving 125.824 g of cobalt(II) nitrate hexahydrate (obtained from Sigma-Aldrich), 2.041 g of ruthenium(III) nitrosyl nitrate (obtained from Alfa Aesar) and 3.381 g lanthanum (III) nitrate hexahydrate (obtained from Sigma-Aldrich) in water. 100 g of Puralox alumina SBA 200 (obtained from Sasol) support, after calcination in air at 750° C. for 2 hours, was impregnated by using one-third of this solution to achieve incipient wetness. The prepared catalyst was then dried in air at 120° C. for 16 hours in a box furnace and was subsequently calcined in air by raising its temperature at a heating rate of 1° C./min to 300° C. and holding it at that temperature for 2 hours before cooling it back to ambient temperature. The above procedure was repeated to obtain the following loading of Co, Ru and La₂O₃ on the support: 20 wt % Co, 0.5% Ru and 1 wt % La₂O₃ and 78.5 wt % alumina.

### Preparation of Hybrid Synthesis Gas Conversion Catalyst Comprising 7.5 wt % Co-0.19 wt % Ru Supported on 73.85 wt % ZSM-5 and 18.46 wt % Alumina (“Catalyst B”)

A catalyst of CoRu (7.5 wt % Co, 0.19 wt % Ru) on ZSM-5 extrudates was prepared by impregnation in a single step. First, ruthenium nitrosyl nitrate was dissolved in water. Second, cobalt nitrate was dissolved in acetone. The volume ratio of the two solutions was similar to the weight ratios of the metals (i.e., 40 acetone:1 water). The two solutions were mixed together and then added to 1/16″ (0.16 cm) extrudates of alumina (20 wt % alumina) bound ZSM-5 zeolite (Zeolyst CBV 014 available from Zeolyst International, having a Si/Al ratio of 40). After the mixture was stirred for 1 hour at ambient temperature, the solvent was eliminated by rotavaporation, also at ambient temperature. Then the catalyst was dried in an oven at 120° C. overnight and finally calcined at 300° C. for 2 hours in a muffle furnace.

### Preparation of Hydrocracking Catalyst Comprising 0.5 wt % Pd Supported on 79.6 wt % ZSM-5 and 19.9 wt % Alumina (“Catalyst C”)

1.305 g of palladium nitrate salt was dissolved in 120 cc of water. The palladium solution was added to 120 g of the same alumina (20 wt % alumina) bound ZSM-5 zeolite described above in the preparation of Catalyst B. The water was removed in a rotary evaporator by heating slowly to 65° C. The vacuum-dried material was dried in air in an oven at 120° C. overnight and finally calcined at 300° C. for 2 hours in a muffle furnace.

### Preparation of Hydroisomerization Catalyst Comprising 1.0 wt % Pt Supported on SSZ-32 (“Catalyst D”)

0.3 g of tetraamine platinum (II) nitrate (obtained from Sigma-Aldrich Company) was dissolved in 20 cc of water. The resulting solution was added to 15 g of SSZ-32 zeolite (prepared according to the method disclosed in U.S. Pat. No. 7,022,308, incorporated herein by reference). Most of the water was removed in a rotary evaporator under vacuum by heating slowly to 65° C. The vacuum-dried material was then further dried in an oven at 120° C. overnight. The dried catalyst was calcined at 300° C. for 2 hours in a muffle furnace.

### Preparation of Hydroisomerization Catalyst Comprising 0.5 wt % Pt Supported on SSZ-32 (“Catalyst E”)

0.15 g of tetraamine platinum (II) nitrate (obtained from Sigma-Aldrich Company) was dissolved in 20 cc of water. The resulting solution was added to 15 g of SSZ-32 zeolite (prepared according to the method disclosed in U.S. Pat. No. 7,022,308, incorporated herein by reference). Most of the water was removed in a rotary evaporator under vacuum by heating slowly to 65° C. The vacuum-dried material was then further dried in an oven at 120° C. overnight. The dried catalyst was calcined at 300° C. for 2 hours in a muffle furnace.

### Activation of Synthesis Gas Conversion Catalyst Ex-Situ

Ten grams each of 20 wt % cobalt-0.5 wt % ruthenium-1.0 wt % lanthanum oxide supported on alumina (conventional Fischer-Tropsch catalyst, Catalyst A) and 7.5 weight % Co-0.19 weight % Ru supported on 72 weight % ZSM-5 and 20 weight % alumina (hybrid synthesis gas conversion catalyst, Catalyst B) catalysts were charged to a glass tube reactor separately. The reactor was placed in a muffle furnace with upward gas flow. The tube was purged first with nitrogen gas at ambient temperature, after which time the gas feed was changed to pure hydrogen with a flow rate of 750 sccm. The temperature of the reactor was increased to 350° C. at a rate of 1° C./minute and then held constant for six hours. After this time, the gas feed was switched to nitrogen to purge the system and the unit was then cooled to ambient temperature. Then a gas mixture of 1 volume % O₂/N₂ was passed up through the catalyst bed at 750 sccm for 10 hours to passivate the catalyst. No heating was applied, but the oxygen chemisorption and partial oxidation exotherm caused a momentary temperature rise. After 10 hours, the gas feed was changed to pure air, the flow rate was lowered to 200 sccm and the temperature was raised to 300° C. at a rate of 1° C./minute and then held constant for two hours. The catalyst was cooled to ambient temperature and discharged from the glass tube reactor.

### Example 1

2.5 grams of Catalyst A and 6.7 grams of Catalyst B were mixed thoroughly and then diluted with 27.6 grams of gamma-alumina. A mixture of 9.4 grams of Catalyst D and 2.25 grams of Catalyst C was transferred to a 316 stainless steel tube reactor of 0.5″ inner diameter and this mixture was placed downstream of the mixed synthesis gas conversion catalysts and separated from them by a small amount of glass wool and gamma alumina beads. The reactor was then placed in a reactor furnace. The catalyst beds were flushed with a downward flow of argon for a period of two hours, after which time the gas feed was switched to pure hydrogen at a flow rate of 400 sccm. The temperature was slowly raised to 120° C. at a temperature interval of 1° C./minute, held constant for a period of one hour, then raised to 250° C. at a temperature interval of 1° C./minute and held constant for 10 hours. After this time, the catalyst beds were cooled to 180° C. while remaining under a flow of pure hydrogen gas. All flows were directed downward.

The dual catalyst beds were then subjected to synthesis conditions in which the catalysts were contacted with synthesis gas at conditions given in Table 1. No additional hydrogen was added. The results of these experiments are shown in Table 1. No solid wax was observed in any of the samples. It can be seen from the results that using a combination of hydrocracking and hydroisomerization catalysts in the downstream bed there is obtained a liquid product free of solid wax under a range of process conditions.

The first set of synthesis rates listed in Table 1 is based on the sum of the weights of the FT catalyst (A) and the hybrid FT catalyst (B). Rates are also listed based on the weight calculated as if all of the cobalt present was at a 20% loading, i.e., five times the cobalt weight. These vary from about 0.5 g(CH₂)/g/h to 0.8 g(CH₂)/g/h for the temperature and pressure range that was covered in Example 1. All of the following examples list rates calculated in this way. As expected, they are constant among the stacked beds at a given condition.

TABLE 1

| sample # | 1 | 2 | 3 | 4 | 5 | 6 | 7 | 8 | 9 | 10 |
|---|---|---|---|---|---|---|---|---|---|---|
| Time on stream (TOS), h | 120 | 311 | 428 | 478 | 646 | 765 | 813 | 861 | 934 | 957 |
| Run Conditions | | | | | | | | | | |
| Temperature, ° C. | 220 | 220 | 220 | 220 | 220 | 225 | 225 | 225 | 225 | 225 |
| Pressure, atm | 10 | 10 | 10 | 10 | 10 | 10 | 15 | 15 | 15 | 20 |
| H₂/CO, nominal | 2 | 2 | 2 | 2 | 2 | 2 | 2 | 2 | 2 | 2 |
| GHSV_{FT}, scc/h/g | 4000 | 6000 | 3000 | 3000 | 2100 | 2100 | 2100 | 3000 | 4000 | 4000 |
| GHSVₜₒₜₐₗ, scc/h/g | 1770 | 2650 | 1320 | 1320 | 930 | 930 | 930 | 1320 | 1770 | 1770 |
| Recycle Ratio | 0 | 0 | 1 | 2 | 1 | 1 | 1 | 1 | 1 | 1 |
| Results | | | | | | | | | | |
| CO Conv, wt % | 40.1 | 26.4 | 46.7 | 46.1 | 60.5 | 74.0 | 79.9 | 61.2 | 45.4 | 50.8 |
| H₂ Conv, wt % | 43.8 | 30.1 | 50.4 | 50.1 | 65.1 | 79.3 | 85.5 | 65.9 | 50.0 | 56.1 |
| Total Conv, wt % | 42.5 | 28.8 | 49.2 | 48.8 | 63.5 | 77.5 | 83.7 | 64.4 | 48.4 | 54.3 |
| Rate, gCH₂/g/h (based on | 0.33 | 0.33 | 0.29 | 0.29 | 0.26 | 0.32 | 0.35 | 0.38 | 0.38 | 0.42 |
| FT + HFT catalyst weights) | | | | | | | | | | |
| Rate, mLC₅₊/g/h (based on | 0.34 | 0.34 | 0.30 | 0.29 | 0.27 | 0.33 | 0.37 | 0.40 | 0.39 | 0.45 |
| FT + HFT catalyst weights) | | | | | | | | | | |
| Rate, gCH₂/g/h (based on 5 | 0.61 | 0.60 | 0.53 | 0.53 | 0.48 | 0.59 | 0.64 | 0.70 | 0.69 | 0.78 |
| times Co weight) | | | | | | | | | | |
| Products | | | | | | | | | | |
| CH₄, wt % | 12.3 | 11.4 | 12.5 | 12.6 | 12.5 | 12.6 | 12.2 | 12.5 | 12.7 | 11.6 |
| C₂ + C₃, wt % | 5.2 | 5.3 | 5.0 | 5.0 | 4.8 | 4.7 | 3.8 | 4.4 | 4.7 | 4.2 |
| C₄, wt % | 5.3 | 5.9 | 5.3 | 5.2 | 5.1 | 4.7 | 3.9 | 4.7 | 5.4 | 4.8 |
| C₅₊, wt % | 76.3 | 76.9 | 76.6 | 76.7 | 77.0 | 77.2 | 79.2 | 77.8 | 76.7 | 79.0 |
| CO₂, wt % | 0.8 | 0.6 | 0.6 | 0.5 | 0.7 | 0.8 | 0.9 | 0.6 | 0.5 | 0.5 |
| C₅ − C₁₀, wt % | 38.2 | 44.9 | 42.2 | 43.0 | 41.0 | 43.6 | 38.9 | 38.2 | 38.7 | 39.0 |
| C₁₁ − C₂₀, wt % | 28.9 | 27.2 | 27.9 | 27.5 | 27.9 | 27.8 | 29.8 | 28.9 | 27.7 | 27.8 |
| C₂₁₊, wt % | 9.2 | 4.8 | 6.5 | 6.2 | 8.1 | 5.9 | 10.6 | 10.7 | 10.3 | 12.0 |
| Isomerized C₂₁₊ paraffin | | 32.9 | | | | 33.7 | | | | |
| concentration, wt % | | | | | | | | | | |
| Cloud point, °C. | | 10 | | | | 2 | | | | |

### Example 2

250 mg of Catalyst A, diluted 50% by weight with gamma-alumina, and 625 g of Catalyst E (0.5% Pd/SSZ-32) were transferred to a 316-SS tube reactor of 5 mm inner diameter. The hydroisomerization catalyst (Catalyst E) was placed downstream of the bed of Catalyst A and separated from it by a small amount of glass wool and gamma alumina beads. The reactor was then placed in a reactor furnace. The catalyst beds were flushed with a downward flow of argon for a period of two hours, after which time the gas feed was switched to pure hydrogen at a flow rate of 400 sccm. The temperature was slowly raised to 120° C. at a temperature interval of 1° C./minute, held constant for a period of one hour, then raised to 250° C. at a temperature interval of 1° C./minute and held constant for 10 hours. After this time, the catalyst beds were cooled to 180° C. while remaining under a flow of pure hydrogen gas. All flows were directed downward.

The catalysts were started up in synthesis gas at 180° C., 10 atm, and H₂/CO of 2 and held at these conditions for several hours to completely saturate the FT catalyst's metal sites, before the temperature was increased to provide CO conversions between 10% and 40% at a flow rate of 50 sccm (12000 cm³/h/g based on the weight of the FT catalyst [catalyst A]). The catalysts were run for over 1200 hours at various temperatures in the range 205° C.-225° C., H₂/CO ratios between 1.5 and 2.0, and pressures from 10 atm to 20 atm. The data collected at the conditions set forth in Table 2 are therefore for a well seasoned set of catalysts. No hydrocracking catalyst was present in the downstream bed. The results are set forth in Table 2. No solid wax was observed in the product at ambient conditions, consistent with the low selectivity to C₂₃+ (less than 3% of the CO converted). It can be seen that at these conditions without the aid of a hydrocracking catalyst such as Pd/ZSM-5 the hydroisomerization catalyst is able to successfully hydrocrack and hydroisomerize Fischer-Tropsch wax to a completely liquid product free of solid wax.

TABLE 2

| | Time on stream, h |
|---|---|
| | 1400 |
| Run Conditions | |
| Temperature, ° C. | 225 |
| Pressure, atm | 10 |
| H₂/CO, nominal | 1.5 |
| Inlet Flow, scc/h | 3000 |
| GHSV_{Total}, scc/h/g | 3430 |
| GHSV_{FT}, scc/h/g | 12000 |
| Results | |
| CO Conversion, % | 25 |
| H₂ Conversion, % | 35 |
| Rate, gCH₂/g_{FT}/h | 0.76 |
| Rate, mL(C₅₊)/g_{FT}/h | 0.75 |
| Products, C % | |
| CH₄, | 12.9 |
| C₂-C₃, | 6.2 |
| C₄, | 6.4 |
| C₅₊ | 74.6 |
| C₅-C₉ | 37.8 |
| C₁₀-C₂₂ | 34.4 |
| C₂₃₊ | 2.4 |

### Example 3

A 5 mm inner diameter reactor tube was loaded with 250 mg of Catalyst A, sized to 125-160 μm, diluted with an equivalent weight of corundum sized to 125-160 μm in the upstream bed in a stacked bed arrangement with 312.5 mg of Catalyst C mixed with 312.5 mg of Catalyst D as the downstream catalyst bed. The catalysts were activated in situ by the procedure described in Example 1.

The dual catalyst beds were subjected to synthesis conditions in which the catalysts were contacted with synthesis gas using a high-throughput screening reactor as supplied by the AG (Heidelberg, Germany). The process conditions and results are set forth in Table 3. No solid wax was observed in the product hydrocarbons at ambient conditions. Note that the total hydrocarbon product rates were 0.5-0.6 g(CH₂)/g/h based on the weight of FT catalyst, and the rate of C₅+ production (72-73% of the total), was also 0.5-0.6 mL/g/h. Selectivity to C₄+ was 80-82%.

TABLE 3

TOS, h

| | 600 | 1150 |
|---|---|---|
| Run Conditions | | |
| Temperature, ° C. | 215 | 220 |
| Pressure, atm | 10 | 10 |
| H₂/CO, nominal | 1.5 | 1.5 |
| Inlet Flow, scc/h | 4000 | 2000 |
| GHSV, scc/h/g_{FT} | 16000 | 8000 |
| Results | | |
| CO Conversion, % | 13.6 | 30.5 |
| Rate, g(CH₂)/g_{FT}/h | 0.54 | 0.59 |
| Rate, mL(C₅₊)/g_{FT}/h | 0.52 | 0.57 |
| Products, C % | | |
| CO₂ | <1 | <1 |
| CH₄ | 11.8 | 13.3 |
| C₂-C₃ | 6.2 | 6.5 |
| C₄ | 9.6 | 7.5 |
| C₅₊ | 72.3 | 72.7 |
| C₅-C₉ | 50.5 | 39.1 |
| C₁₀-C₂₂ | 21.7 | 31.8 |
| C₂₃₊ | 0.1 | 1.8 |

### Example 4

ZSM-12 zeolite powder was first calcined at 550° C. for 2 hours. 50 g of the calcined ZSM-12 powder and 12.5 g of catapal B alumina powder were added to a mixer and mixed for 10 minutes. 30.6 g of deionized water and 0.89 g of nitric acid were added to the mixed powder and mixed for 10 minutes. The mixture was then transferred to a 1 inch (2.54 cm) BB gun extruder available from The Bonnot Company (Uniontown, Ohio) and extruded through a dieplate containing forty-eight 1/16″ (0.16 cm) holes. The ZSM-12 extrudates were dried first at 70° C. for 2 hours, then at 120° C. for 2 hours and finally calcined in flowing air at 600° C. for 2 hours.

1.305 g of palladium nitrate salt was dissolved in 120 cc of water. The palladium solution was added to 120 g of alumina (20 wt % alumina) bound ZSM-12 zeolite prepared as described above. The water was removed in a rotary evaporator by heating slowly to 65° C. The vacuum-dried material was dried in air in an oven at 120° C. overnight and finally calcined at 300° C. for 2 hours in a muffle furnace.

A 5 mm inner diameter tube reactor was loaded with 250 mg of Catalyst A, sized to 125-160 μm, diluted with an equivalent weight of corundum sized to 125-160 μm in the upstream bed and loaded in a stacked bed arrangement with 500 mg of the 0.5 wt % Pd/ZSM-12 hydrocracking catalyst described above as the downstream catalyst bed. The catalysts were activated in situ by the procedure described in Example 1.

The dual catalyst beds were subjected to synthesis conditions in which the catalyst was contacted with synthesis gas using a high-throughput screening reactor as supplied by the AG (Heidelberg, Germany). The process conditions and results are set forth in Table 4. No solid wax was observed in either of the samples at ambient conditions.

TABLE 4

sample #

| | |
|---|---|
| 1 | 2 |

TOS, h

| | 840 | 1273 |
|---|---|---|
| Run Conditions | | |
| Temperature, ° C. | 220 | 225 |
| Pressure, atm | 10 | 15 |
| H₂/CO, nominal | 1.5 | 1.5 |
| Inlet Flow Rte, scc/h | 4000 | 4000 |
| GHSV, scc/h/g_{FT} | 16000 | 16000 |
| Results | | |
| CO Conversion, % | 21 | 19 |
| Rate, gCH₂/g_{FT}/h | 0.61 | 0.69 |
| Rate, mLC₅₊/g_{FT}/h | 0.57 | 0.62 |
| Products, C % | | |
| CH₄ | 14.5 | 16.6 |
| C₂-C₃ | 7.0 | 7.4 |
| C₄ | 8.8 | 9.4 |
| C₅₊ | 69.7 | 66.7 |
| C₅-C₉ | 42.0 | 42.3 |
| C₁₀-C₂₂ | 27.1 | 24.0 |
| C₂₃₊ | 0.5 | 0.5 |

### Example 5

A 5 mm inner diameter tube reactor was loaded with a physical mixture of 125 mg of Catalyst A and 250 mg of Catalyst B, both sized to 125-160 μm in the upstream bed and loaded in a stacked bed arrangement with 300 mg of Catalyst E as the downstream catalyst bed. The catalysts were activated in situ by the procedure described in Example 1.

The dual catalyst beds were subjected to synthesis conditions in which the catalysts were contacted with synthesis gas using a high-throughput screening reactor as supplied by the AG (Heidelberg, Germany). The process conditions and results are set forth in Table 5. No solid wax was observed in the sample at ambient conditions. Rates were calculated based on a weight 5 times the weight of cobalt present, as if all of the synthesis component contained cobalt at a 20 wt % level. The rates per unit weight of cobalt are the same as those for Example 2. The C₅+ selectivity was 72-74% and the C₄+ selectivity 80-82% under the conditions shown.

TABLE 5

Time On Stream, h

| | 600 | 1158 |
|---|---|---|
| Run Conditions | | |
| Temperature, ° C. | 215 | 220 |
| Pressure, atm | 10 | 10 |
| H₂/CO, inlet | 1.5 | 1.5 |
| Inlet Flow, scc/h | 4000 | 2000 |
| GHSV, ssc/h/g_{(A+B)} | 10667 | 5333 |
| Results | | |
| CO Conversion, % | 13.4 | 28.0 |
| Rate, g(CH₂)/g_{FT}/h | 0.62 | 0.62 |
| Rate, mL(C₅₊)/g_{FT}/h | 0.60 | 0.60 |
| Products, C % | | |
| CO₂ | 0.2 | 0.5 |
| CH₄ | 11.9 | 13.4 |
| C₂-C₃ | 6.2 | 6.3 |
| C₄ | 7.3 | 7.6 |
| C₅₊ | 74.4 | 72.2 |
| C₅-C₉ | 39.9 | 41.7 |
| C₁₁-C₂₂ | 31.6 | 28.5 |
| C₂₃₊ | 2.9 | 2.0 |

### Comparative Example

250 mg of Catalyst A, diluted 400% by volume with gamma-alumina was transferred to a 316-SS tube reactor of 5 mm inner diameter. Silica, an inert filler, was placed downstream of the bed of Catalyst A and separated from it by a small amount of glass wool and gamma alumina beads. The reactor was then placed in a reactor furnace. The catalyst beds were flushed with a downward flow of argon for a period of two hours, after which time the gas feed was switched to pure hydrogen at a flow rate of 400 sccm. The temperature was slowly raised to 120° C. at a temperature interval of 1° C./minute, held constant for a period of one hour, then raised to 250° C. at a temperature interval of 1° C./minute and held constant for 10 hours. After this time, the catalyst beds were cooled to 180° C. while remaining under a flow of pure hydrogen gas. All flows were directed downward.

The catalyst was then started up as described in Example 2. After the catalyst was aged for 1200 hours, data were collected for the synthesis conditions set forth in Table 6. No hydrocracking or hydroisomerization catalyst was present in the downstream bed. The results are also set forth in Table 6.

It can be seen that the activity decline, over the period from 600 hours to 1150 hours on stream, was almost exactly made up by a 5° C. increase in operating temperature for both Example 5 and Example 6. Synthesis rates for the Fischer-Tropsch catalyst alone (Catalyst A) were about 0.6 g(CH₂)/g/h, the same as those measured for Example 2 and Example 5. 8-11% of the converted CO went to C₂₃+ hydrocarbons over Catalyst A at these conditions. That is too much to be retained in solution at ambient temperature, thus it formed a separate wax phase. The 2-3% C₂₃+ made in Example 2 and Example 5 stayed in solution in the rest of the C₅+ product at room temperature and would not separate unless the product were cooled to 10° C. or lower.

Catalyst A produced 80-82% C₄+ hydrocarbons and 73-76% C₅₊ hydrocarbons, whereas the catalyst systems of Example 2 and Example 5 also made 80-82% C₄+, but slightly less C₅+ (72-73%, 72-74%). Thus the systems of Example 2 and Example 5 produced about 1% more C₄ hydrocarbons, but very little additional C₁-C₃. The C₅+ liquid products were lighter for Example 2 and Example 5, as about 40% of the CO conversion went to the C₅-C₉ fraction, compared with only 31% for catalyst A alone. Most of the increase came from cracking of the waxes, and some (3-4%) came from net cracking of the diesel range products.

TABLE 6

Time on stream, h

| | 600 | 1150 |
|---|---|---|
| Run Conditions | | |
| Temperature, ° C. | 215 | 220 |
| Pressure, atm | 10 | 10 |
| H₂/CO, nominal | 1.5 | 1.5 |
| Flow, scc/h | 4000 | 2000 |
| GHSV_{FT}, scc/h/g | 16000 | 8000 |
| Results | | |
| CO Conv, wt % | 15 | 31 |
| H₂ Conv, wt % | 21 | 43 |
| Total Conv, wt % | 18 | 38 |
| Rate, gCH₂/g/h | 0.59 | 0.61 |
| Rate, mLC₅₊/g/h | 0.57 | 0.59 |
| Products, C % | | |
| CO₂ | <1 | 0.8 |
| CH₄ | 11 | 13 |
| C₂ + C₃ | 6.5 | 7 |
| C₄ | 6.5 | 6.5 |
| C₅₊ | 76 | 73 |
| C₅-C₉ | 31 | 31 |
| C₁₀-C₂₂ | 34 | 33 |
| C₂₃₊ | 11 | 8.5 |

What is claimed is:

1. A process for converting synthesis gas to a hydrocarbon mixture comprising contacting a feed comprising a mixture of carbon monoxide and hydrogen with a synthesis gas conversion catalyst in an upstream bed and a hydroisomerization catalyst containing a metal promoter and an acidic component in a downstream bed downstream of the upstream bed within a single reactor, such that C₂₁₊ normal paraffins are formed over the synthesis gas conversion catalyst and said C₂₁₊ normal paraffins are hydroisomerized over the hydroisomerization catalyst, thereby resulting in a hydrocarbon mixture containing no greater than 5 weight % C₂₁+ normal paraffins.

2. The process of claim 1 wherein the upstream bed and the downstream bed have an essentially common reactor temperature and an essentially common reactor pressure.

3. The process of claim 1 wherein the synthesis gas conversion catalyst comprises cobalt on a solid oxide support.

4. The process of claim 3 wherein the solid oxide support is selected from the group consisting of alumina, silica, titania and mixtures thereof.

5. The process of claim 1 wherein the synthesis gas conversion catalyst comprises cobalt supported on an acidic component.

6. The process of claim 1 wherein the synthesis gas conversion catalyst comprises a mixture of cobalt on a solid oxide support and cobalt supported on an acidic component.

7. The process of claim 1 wherein the hydroisomerization catalyst comprises a zeolite of the SSZ-32 family.

8. The process of claim 1 wherein the downstream bed further comprises a hydrocracking catalyst selected from the group consisting of amorphous silica-alumina, tungstated zirconia, zeolitic crystalline medium pore molecular sieve and non-zeolitic crystalline medium pore molecular sieve.

9. The process of claim 1 wherein the hydroisomerization catalyst further comprises a metal promoter selected from the group consisting of cobalt, nickel, copper, ruthenium, rhodium, rhenium, palladium, silver, osmium, iridium, platinum, gold, molybdenum, tungsten, and oxides, and combinations thereof.

10. The process of claim 8 wherein the hydrocracking catalyst further comprises a metal promoter selected from the group consisting of cobalt, nickel, copper, ruthenium, rhodium, rhenium, palladium, silver, osmium, iridium, platinum, gold, molybdenum, tungsten, and oxides, and combinations thereof.

11. The process of claim 1 wherein the reactor temperature is between about 160° C. and about 260° C.

12. The process of claim 1 wherein the reactor temperature is between about 175° C. and about 250° C.

13. The process of claim 1 wherein the reactor temperature is between about 185° C. and about 235° C.

14. The process of claim 1 wherein the temperature of the first catalyst bed and the temperature of the second catalyst bed differ by no more than about 20° C.

15. The process of claim 1 wherein the synthesis gas conversion catalyst further comprises a promoter selected from the group consisting of ruthenium, rhenium, platinum, palladium, gold, and silver.

16. The process of claim 1 wherein the hydrocarbon mixture produced comprises:
0-20 weight % CH₄;
0-20 weight % C₂-C₄; and
60-95 weight % C₅₊.

17. The process of claim 1 wherein the gaseous hourly space velocity is between about 100 and about 5000 volumes of gas per volume of catalyst per hour.

18. The process of claim 1 wherein the reactor pressure is between about 3 atmospheres and about 35 atmospheres.

19. The process of claim 1 wherein process water is not separated from the reactor during the hydroisomerization of said C₂₁₊ normal paraffins.

20. The process of claim 1 wherein no hydrogen in addition to the mixture of carbon monoxide and hydrogen is added to the reactor.

21. The process of claim 1 wherein the hydrocarbon mixture is substantially free of solid wax at ambient conditions.

22. The process of claim 1 wherein the hydrocarbon mixture has an isomerized C₂₁₊ paraffin concentration of at least 30 weight % based on the weight of the C₂₁₊ fraction.

23. A process for converting synthesis gas to a hydrocarbon mixture comprising contacting a feed comprising a mixture of carbon monoxide and hydrogen with a synthesis gas conversion catalyst in an upstream bed and a hydroisomerization catalyst containing a metal promoter and an acidic component in a downstream bed downstream of the upstream bed within a single reactor at an essentially common reactor temperature and an essentially common reactor pressure, such that C₂₁₊ normal paraffins are formed over the synthesis gas conversion catalyst and said C₂₁₊ normal paraffins are hydroisomerized over the hydroisomerization catalyst, thereby resulting in a hydrocarbon mixture having a cloud point no greater than 15° C.

24. The process of claim 23 wherein the hydrocarbon mixture contains no greater than 5 weight % C₂₁₊ normal paraffins.
